(19) United States
(12) Patent Application Publication
(10) Pub. No.: US 2008/0268515 A1
(43) Pub. Date: Oct. 30, 2008

(54) Method and apparatus for production and refinement of microbial consortia for the generation of selective therapeutic chemical agents

(76) Inventors: Roy Cullimore, Regina (CA); Charles R. Pellegrino, Long Beach, NY (US); Lori Johnston, Regina (CA); Mark Newman, Port Jefferson Station, NY (US)

Correspondence Address: BRUCE L. ADAMS, ESQ.;ADAMS & WILKS, SUITE 1231, 17 BATTERY PLACE, NEW YORK, NY, 10004, US

(21) Appl. No.: 12/156,240

(22) Filed: May 29, 2008

Related U.S. Application Data
(63) Continuation-in-part of application No. 10/812,436, filed on Mar. 30, 2004.

(51) Int. Cl. C12N 11/00; C12M 1/00

(52) U.S. Cl. 435/174; 435/289.1

(57) ABSTRACT

A culturing fluid of defined bacterial consortia is placed within a cultivation apparatus to generate growths of either a bioconcretious or biocolloidal form within, or attached to surfaces exposed to, the culturing fluid. Such growths involve the interactive activities between all of the bacterial consortia to generate growths and economically attractive chemical daughter and end products within the fluid environment bounded by the cultivation apparatus. The cultivation apparatus allows the culturing fluid to flow along passageways that have alternating constricted and expanded zones in a manner that creates the growth of bioconcretious or biocolloidal structures within which the bacterial consortia interact to allow maintenance of these growth structures and stimulate the production of the desired chemical products of significance. The bacterial consortia generate either biocolloids or bioconcretions within an electrically charged field that is generated by the interaction of the consortia with dissimilar metal or carbon surfaces of the passageways. The grown interacting bacterial consortia and their culturing fluids can be used as sources of chemicals such as therapeutic agents that are anti-microbial, probiotic, anti-biotic, or anti-cancer agents.

### CROSS REFERENCE TO RELATED APPLICATION

This application is a continuation-in-part of copending application Ser. No. 10/812,436 filed Mar. 30, 2004, the contents of which are incorporated herein by reference in their entirety.

### BACKGROUND OF THE INVENTION

1. Field of the Invention

The present invention relates to a method and apparatus that would be generally applied in the formation, culture and use of microbial consortia to generate various forms of bioconcretions and, more particularly, to the production of a mass culture of a consortia for the purpose of generating a desired product from within the bioconcretion that, by the use of methods that are appropriate for the optimization and refinement of the determined product, could have use as an anti-microbial, anti-cancer, or otherwise biochemically significant function.

2. Discussion of the Related Art

Advances in the field of microbiology have largely been based upon the premise that single species of microorganisms are capable of efficiently producing useful products for humankind. These products have ranged from organic solvent and selected biochemical agents, to antibiotics and plasmids. In all of these developments, there has been a preoccupation with the utilization of a single species, commonly with refinement using common agar plate technologies, leading to the development of single specific chemical compounds. Heretofore, virtually no scientific attention has been paid to the use of complexes of bacterial consortia within bioconcretious or biocolloidal habitats to either generate a defined product or compete successfully with nuisance or pathogenic microorganisms or viral particles.

The scientific community has generally overlooked the synergistic activities of species within consortia to achieve sophisticated products, conditions and effects. Consortia may be defined as “an association esp. of several business companies” (Concise Oxford Dictionary, 8th edition, 1991). These species consort in a manner that harmonizes their activities for the benefit of the community. Cullimore (Cullimore, D. R. (2000), “Practical Atlas for Bacterial Identification”, Lewis Publishers & CRC Press, Baton Rouge, Fla.; pages 131 to 136) proposed an identification scheme for consortia that was based upon the reaction pattern signatures obtained by bacteriological testing using the biological activity reaction test (BART™, Droycon Bioconcepts Inc, Regina, Canada). Cullimore et al. (Cullimore, D. R. and L. Johnston “The Impact of Bioconcretious Structures (Rusticles) on the RMS Titanic: Implications to Maritime Steel Structures”, Proceedings of the Annual Meeting Technical sessions of the Society of Naval Architects and Marine Engineers, October, 2000, Vancouver, Canada; pages 9-1 to 9-16) specifically defined the bacterial communities that were found to form a part of the microbial consortia that were associated with iron-rich bioconcretions (commonly know as rusticles) growing on the steel of the shipwreck RMS Titanic and could be cultured in the laboratory. These findings were in direct contradiction to the findings of Iverson (Iverson, W. P. (2001), “Research on the mechanisms of anaerobic corrosion”, International Biodeterioration and Biodegradation (47); 63-70) showing that “stalactite” growths could be obtained in the presence of Desulfovibrio and a potential influence of electrical potentials. However the conditions of this study did not deliberately encourage the presence of microbiologically influenced consortia.

One feature of many consortia is that they commonly form bioconcretious structures around the community structures as functional protective barriers to invasion and predation by alien species. For the rusticle-forms of bioconcretion this is described by Cullimore et al. (Cullimore, D. R., Pellegrino, D. and L. Johnson (2002), “RMS Titanic and the emergence of new concepts on consortial nature of microbial events”, Rev Environ Contam Toxicol 173:117-141). Such bioconcretions are usually predominantly crystalline in form with a high porosity and many pathways and portals to allow the movement of water, gases, nutrients and waste products through the structure. The method and apparatus described herein address primarily the ability of such microbial consortia within a defined bioconcretion to collectively generate chemical compounds or agents that would protect the consortia from detrimental infestations of the bioconcretion by alien microorganisms. It is an implicit part of the method and apparatus that such collectively synthesized compounds or agents would only be generated as a result of the cooperative action between the bacterial communities associating within the consortia.

This approach historically contravenes the standard scientific practices that have been held as the norm. For instance, microbiology has pursued a pathway developed by other natural sciences and, in particular, zoology, botany and chemistry. From the latter three disciplines involved in natural sciences, then the concept was adopted to separate and study microbial species in much the same manner as plants, animals and even chemical molecules as independent entities in a manner that would lead to the definition by division of species of plants and animals; as well as the differentiation of chemicals. Using the accepted disciplines employed for plants and animals, the differentiation of microorganisms has been achieved at descriptive, cultural, biochemical and molecular levels. This combination of interest foci (at the species and molecular levels) has led to a lack of development in the understanding of microbial communities and the manner with which these consortia are able to collectively generate complex bioconcretions along with specific agents of interest. As a consequence of the acceptance of standard microbiological practices involving agar cultural techniques and traditional broth techniques, scientific discoveries have been limited to those that are directly attributable to single-species events often commonly reproducible cultural technologies. In view of the foregoing shortcomings of the prior art expressly for the methods and apparatus for the mass culture of consortia, these methods are presented as unique vehicles for the generation of therapeutic or otherwise useful agents.

At its most fundamental level, the nature of most microbial activity occurs through the common practice of microbial species co-existing mutualistically in communities commonly referred to as consortia. These consortia therefore function as integrated populations possessing the common goals of sustenance, survival and dominance over alien biota within their particular environment. Such activities involve cooperation between the species that can extend towards a synergy in which the total product exceeds the sum of the contributions of each of the component species. This synergy extends towards improved structural supports for the community such as through the synthesis of ferric oxides and hydroxides (such as goethites) and carbonates (such as hematites, siderites and dolomites). Aspects of the formation of complex consortial forms of growth was described by Cullimore et al., 2002, with respect to the forms of iron rich bioconcretious porous growths that occur on deep ocean steel fabricated shipwrecks. These are commonly referred to as rusticles.

Consortia commonly involved in rusticles have been found to be formed at separate locations but function co-operatively. Cullimore et al. (Cullimore, D. R. and L. Johnson (2005), “Learning from tragedies, what is colonizing the Titanic today?”, Voyage 51:109-122) described the different communities involved that were detected using the BART tester systems described by Cullimore, 1999 (Cullimore, D. R. (1999), “Microbiology of well biofouling”, Lewis Publishers CRC Press, Baton Rouge, Fla.; pages 137-140, 262-280). Major bacterial consortia included the iron related, IRB; sulfate reducing, SRB; slime forming, SLYM; heterotrophic, HAB; and denitrifying bacteria, DN; bacteria. These bacterial consortia, along with various fungal species, were found to collectively generate the bioconcretious growths. A feature unique to these observations was that these bioconcretial growths could, using the method and apparatus of the present invention, generate specific products that, when assayed, possessed unique antibacterial or anticancer properties. Vinebrooke et al. (Vinebrooke, R. D. and Cullimore, D. R. (1998), “Natural organic matter and the bound water concept in aquatic systems”, Rev Environ Contam Toxicol 155:111-127) reported that bacterial consortia can also form within biologically driven colloidal structures in various waters with the production of useful products as defined above. The premise for the methodology and apparatus therefore extends to any self-sustaining microbiologically influenced complex consortial structures which posses the various means for retaining and transporting water, including plate-like shells, bacterial threads and fibrillar bundles, all within a liquid saturated common porous solid or colloidal material influenced by microbiological activities. The present invention is particularly concerned with the method and apparatus that would allow the microbiologically influenced production of specific agents from consortial growths generated within naturally enhanced or industrial constructions.

In the decade from 1996 to 2006, microbiological investigations have revealed that biocolloidal- or bioconcretion-based growths involve a complex of microbial consortia that can generate the recognized and desired product through cooperative synthetic actions involving contributions from the various living components within the consortia. This invention therefore utilizes the unique findings of these consortial activities using an apparatus and method that are unique in their design and applications.

### SUMMARY OF THE INVENTION

The present invention relates to the synthesis of microbial consortia within an apparatus that would enable sustained production of a mass consortia culture by applying a methodology that would allow the consistent generation of the desired defined product of that cooperative consortial activity. Such desired products could range from antibiotics, to anticancer agents to specific chemical components that have unique desirable properties such as functionally through formation within the consortially influenced structures. Some of these products from cooperative consortial activities could also have value in the construction and chemical industries requiring exceptional bonding properties.

Accordingly, the present invention relates to an apparatus and method wherein administered consortia of microorganisms interactively generate properties and products of definable and reproducible form that have significant value to society. To be effective, each consortium involves a plurality of strains of microorganisms in which the collective action and interactions of these strains generate properties and/or products that do not occur without the presence of the plurality of strains and their associated activities with other implanted consortia within the managed system. In accordance with the present invention, collections of multiple strains of microorganisms, referred to herein as “consorms” or “consortia”, are artificially managed to generate cultures that would generate agents or unique procedures of value when extracted. In particular, the present invention relates to various aspects of the apparatus and methodologies for the blended production and use of defined microbial consortia which would include the production of mass cultures of the blended combination of specific consortia that would then over time generate a desired product or process.

For those familiar with the art, it may also be considered that a probable evolutionary trend could now allow one or more solitary species to generate a specific chemical defense (as an example of a desired product), even if most products are constructed via more complex synergistic activities between different consortial species. Prolonged adaptation could now allow a single species to mimic part of the collective potential of the consortia and subsequently this species could generate within the apparatus and method such desired product as had previously been sustained by the consortia. Such a species would have to have adapted from within the consortia to exploit such essential parts of the activities that could now allow the single species to mimic the collective potential of the consortia. Such an event may be considered to be a result of the defined species having evolved from within the consortia through the exploitation and adoption of the significant cellular mechanisms that had previously been bestowed to the consortia by various species within the consortia via more complex synergistic activities functioning in a concerted and cooperative manner. This apparatus and methodology therefore includes the potential that the desired product or process could be a part of a product from, or the fully generated and complete consortial structure.

Alternately, and depending upon its efficacy, the desired product could be refined from the consortial structure or soluble/particulate/colloidal products released by a consortium during stages of growth and/or processing. This may include viable or inanimate cells from some or all of the species inherently a part of the interacting consortia. It may also be considered an inevitable evolutionary trend for a single species from within the consortia to generate overtime, through the processes of natural adaptation, the ability to generate and manipulate all of the functions and properties associated with the formation of the desired product without the need for the activities of other members of the consortia from which this species evolved incorporating within the domain of the single species all of these enhanced and desirable abilities thus rendering the need for consortially driven synthesis of the product no longer valid. This apparatus and methodology therefore includes the potential for the consortia so cultured to generate a single species of microorganisms now able to undertake all of the desired functions that previously required the participation of all active members of the consortia.

Within the apparatus one of the distinctive features is the application of an impressed electrical charge to surfaces and colloidal entities placed as a part of the methodology within the apparatus that would cause the unique focusing of microbiological consortial activities in a manner to entrap the products or processes of that activity in a recoverable manner within liquid, semi-saturated and/or saturated porous media. Within the methodology, the identification and culture of essential bacterial consortia to the effectiveness of the process are the patented Biological Activity Reaction Test (“BART”) systems, described by Cullimore, 1999 and 2000, and these systems were used to identify the products of interest (e.g., antibiotics), optimize the production process, define the methodology for refinement and conduct the necessary quality control programs inherent in a well managed manufacturing and pharmaceutical plant.

### BRIEF DESCRIPTION OF THE DRAWINGS

FIG. 1 is an explanatory view of an exemplary embodiment of an apparatus according to this disclosure configured in a manner that allows the focused growths of bioconcretious or biocolloidal structures and assures effective cooperative activities of the bacterial consortia selected and applied within the culturable fluid zone of the apparatus;

FIG. 2 is a diagrammatic view of the apparatus, partly in section, indicating the management of fluids being administered to the apparatus for the generation of bioconcretious and biocolloidal structures;

FIG. 3 is an enlarged schematic view of a portion of the apparatus in which selected bacterial consortia are administered in a preordained sequence along with nutrients and culturable fluids to effect a bioconcretious growth within which all of the applied bacterial consortia become integrated as distinct components within the synthesized structure; and

FIG. 4 is an enlarged schematic view of a portion of the apparatus in which selected bacterial consortia are administered in a preordained sequence along with nutrients and culturable fluids to effect a biocolloidal growth within which all of the applied bacterial consortia become integrated as distinct components within the synthesized structure.

### DETAILED DESCRIPTION OF THE INVENTION

The present invention broadly relates to an apparatus and method through which the general ability of the applied microbial consortia within the apparatus and using the unique combination of methodologies to collectively produce in a cooperative manner such products and chemical compounds that have either therapeutic or commercial value. The following description of the apparatus and methodology for the effective activities of combinations of defined selected bacterial consortia to generate growth structures within, or around which, various desirable chemicals or chemical agents can be synthesized within the fluid environment within the apparatus. As will be appreciated by those skilled in the art, the invention involves the cooperative interaction of bacterial consortia to primarily form a solid (bioconcretious) or semi-solid (biocolloidal) structure, and secondarily for that structure to then generate daughter or end chemical products that are found to have significant value to society.

The following detailed description is provided in two sections: (1) Apparatus, and (2) Methodology. In combination, the unique features of the apparatus and method allow various elected methods through which the designated bacterial consortia can initiate an integrated form of growth within either a bioconcretious or a biocolloidal structure. In this disclosure, the growing structures from the combined activities of the bacterial consortia occur because of the manner of the flow of designated culturing fluids through the apparatus to create either bioconcretious or biocolloidal structures. Through the application of the selective cultural methodologies, it now becomes possible to create either bioconcretious or biocolloidal structures that during their natural life span are able to generate economically significant chemical products such as antibiotics and anticancer agents.

### 1. APPARATUS

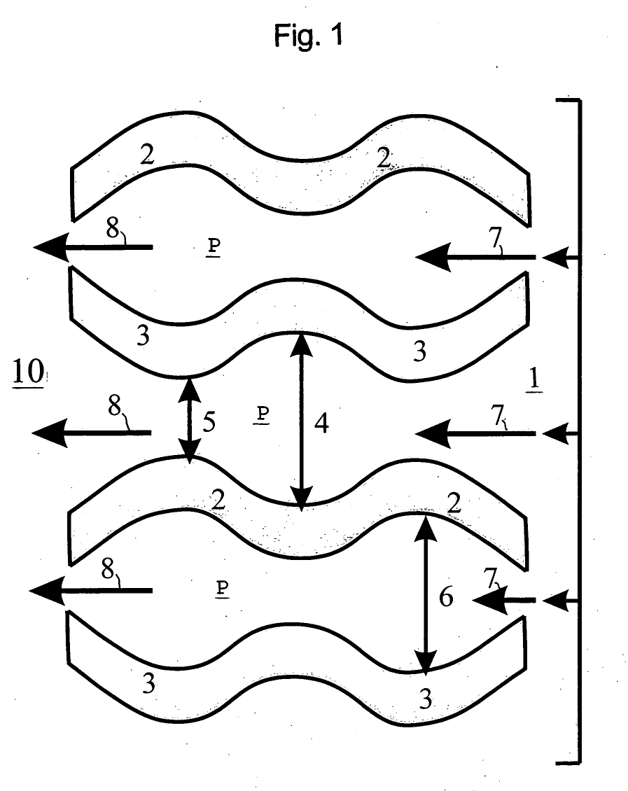

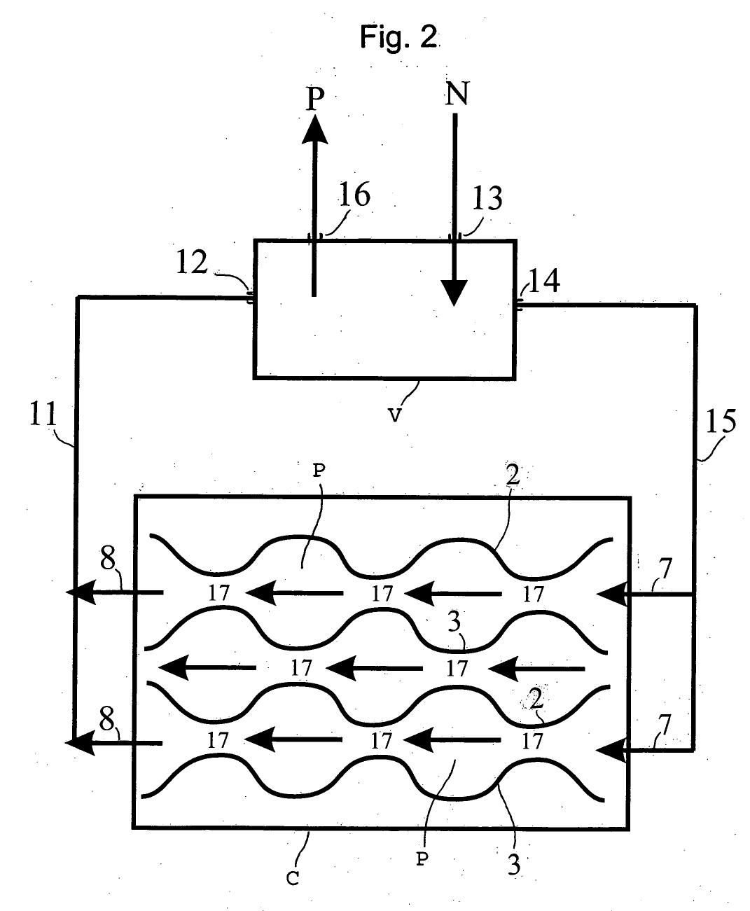

One example of an apparatus for growing bioconcretious and biocolloidal structures according to this disclosure is illustrated in FIGS. 1-4. In its simplest form, the apparatus comprises a growing chamber C having flow passageways through which a culturing fluid flows to grow bioconcretious or biocolloidal structures within the culturing fluid and on surface portions of the flow passageways. As shown in FIGS. 1-2, the growing chamber C has an inlet side 1 for admitting a culturing fluid of bacterial consortia into the growing chamber, and an outlet side 10 for discharging the culturing fluid from the growing chamber. A plurality of flow passageways P are connected in parallel flow relationship between the inlet side 1 and the outlet side 10 of the growing chamber C. In this embodiment, the growing chamber C is comprised of rectangular plastic walls though other suitable shapes and materials may be employed.

The flow passageways P are each formed of two opposed, space-apart walls or partitions 2 and 3 which define therebetween the flow passageway P. Preferably, each two adjoining flow passageways P share a common wall 2 or 3, as illustrated in this embodiment. The walls 2 and 3 extend from one sidewall of the growing chamber C to the other sidewall thereof, i.e., in a direction perpendicular to the plane of the paper, and have a wavy or undulating shape in the flow direction of the culturing fluid (denoted by arrows) from the inlet side 1 to the outlet side 10. The undulations of the wall 2 are arranged relative to the undulations of the wall 3 to form alternate constricted and expanded flow regions 5 and 4 along the lengths of the flow passageways. As shown in FIG. 1, the opposed walls 2, 3 of each flow passageway P are alternately spaced closer to and farther from one another to define the alternate constricted and expanded flow regions 5 and 4.

As shown in FIG. 2, the growing chamber C is connected in a closed fluid loop with a control vessel V. A collection conduit 11 is connected at one end to the outlet side 10 of the growing chamber C and connected at its other end to an inlet 12 of the control vessel. An outlet 14 of the control vessel V is connected to one end of a return conduit 15, and the other end of the return conduit is connected to the inlet side 1 of the growing vessel C. A circulator pump (not shown) may be included in the closed fluid loop to circulate the culturing fluid around the closed loop. The control vessel V has an outlet port 16 for extracting a portion of the culturing fluid and an inlet port 17 for introducing additives and supplemental culturing fluid into the control vessel. Though not illustrated, the apparatus includes equipment and controls (pumps, valving, temperature regulator, gauges, monitors and the like) suitable to carry out the methodologies described herein, as would be readily recognized and easily implemented by persons of ordinary skill in the art.

To maximize the potential for the generation of the growth of biological structures from the selected bacterial consortia being cultured within the apparatus, the opposed walls or partitions 2 and 3 of the flow passageways P are preferably made of two types of sheets 2 and 3 that are configured in a harmonic pattern and are composed of materials that are different in composition and fabricated from either metal (including metal alloys) or carbon of known porosities and surface structures that would, through their interactive and unique forms, create the desired biological growth structures within the flowing culturing fluids and attachments to the sheet surfaces.

By way of example, the opposed walls 2 and 3 of the flow passageways may be constructed of different metal alloys such as mild steel and an aluminum-zinc alloy. These dissimilar metals will generate, when infested with the applied bacterial consortia, low levels of naturally induced electrical charges measurable in the 25 to 800 millivolt range. Such consortially manipulated charges create a natural electrolytic function that causes a focusing of bioconcretious growths around impressed anodic sites on the walls of the flow passageways. The opposed walls 2 and 3 may also be constructed of layers of porous carbon in which case the introduced bacterial consortia will penetrate into the carbon pores and create electrical potentials within the carbon surfaces that would result in charges across from the opposed walls of 50 to 1,050 millivolts. Within these fields biocolloidal structures will form provided that the forward flow of culturing fluid across the walls is less than 2 mm per second.

The harmonic form created within the two sheets are comparable in geometry but reversed in phase. As shown, the different sheets 2 and 3 have a constantly changing distance between them, varying from a maximal distance 4 to a minimal distance 5 as the culturing fluid flows laterally through the growing chamber C. In this embodiment, the maximal distance 4 is preferably 8 mm and the minimal distance 5 is preferably 5 mm, and these distances have been greatly exaggerated in the drawings for clarity of illustration. These varying distances set up a number of effects that can cause the form of the bacterial consortial structures to be generated within the growing chamber. These effects include the creation of turbulent and compressive flows, and constant changes in the fluid flow rates at any location, such as at location 6, cause the focusing of interactive bacterial consortial locations and subsequent growth form and structure as the culturing fluid flows (arrows 7) between the paired harmonic sheets 2 and 3 along and then exits the growing chamber (arrows 8) for recycling as disclosed in FIG. 2.

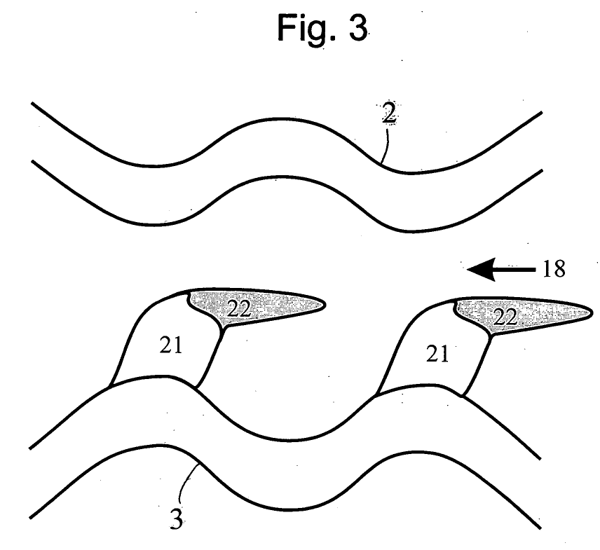

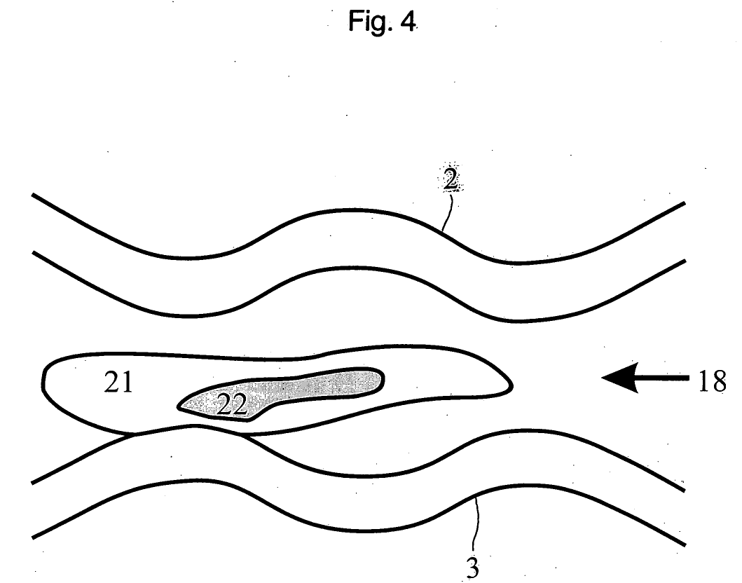

FIG. 2 shows a vertical section through the apparatus in which the lateral flows of culturing fluid (arrows 8) are collected in the collection conduit 11 and delivered to the inlet 12 of the control vessel V. A portion of the culturing fluid is removed from the control vessel V through the outlet port 16 for the extraction and concentration of the desired product P followed by the addition of additives N of chemical factors and modification of any biological, chemical and/or physical factors (for example, water, pH, oxidation-reduction potential modifiers, temperature, flocculants, additional blended bacterial consortial suspensions, nitrogen) to optimize the continued effective production of the apparatus. The culturing fluid, altered by the removal of product and supplemented with additives, flows out of the outlet 14 of the control vessel V and is returned by the return conduit 15 to be split and injected directly into the inlet side 1 of the growing chamber C so that the culturing fluid flow goes through a series of compressive events 17 as the fluid passes through the sites where the opposed walls 2 and 3 are in closest proximity. Flows created through the apparatus are dependent upon the levels of supplements added to the culturing fluid with a faster production flow being designed to create bioconcretious growths (see FIG. 3) and a slower flow to create biocolloidal growths (see FIG. 4).

In general, the growing chamber C has a total fluid volume in which the fluid volume within the flow passageways P would occupy 50±10% and the fluid volume associated with the upper and lower walls of the growing chamber would occupy the remaining 50±10% with the movements of recycling fluids within the apparatus being determined by the form of growths designated by the applications of consortia, nutrients, chemicals and management of physical factors such as pH, oxidation reduction potential and hydraulic flow. Recycling of the total fluid volume would be at a rate of less than 10±5% for the formation of biocolloids and greater than 15±5% for bioconcretions.

Typical bioconcretious growth structures are illustrated in FIG. 3, which shows a vertical cross-section of a portion of the growing chamber with culturing fluid flow coming from the right (arrow 18) and moving laterally through the growing chamber between the two opposed walls 2 and 3 disposed to form a flow passageway having constantly varying throat widths for the fluid flowing therethrough. Electrical charges are created through the natural impression of the metal alloy or carbon surfaces resulting from the attachment and growth activities of the integrated bacterial consortia implanted within the apparatus and generating concretions growths 21 commonly forming at the narrowest points (throats) in the flow passageways and extending upwards and towards the source of fluid flow, as shown by reference numeral 22. These growths gradually occlude the movement of fluids and this is addressed in the methodology section.

The generation of bioconcretious structures involves the inoculation of the apparatus with the defined bacterial consortia in a process that allows effective interactions to occur under suitable conditions including the application of chemical feeds to assure the formation of the bioconcretion with stimulation by such chemicals as ferrous iron, calcium and carbon dioxide to allow effective synthesis of the desired growth at sites created by the natural impression of anodic charges into the walls 2, 3 of the flow passageways P.

FIG. 4 illustrates the position of biocolloids generated by the integrated and managed activities of the selected bacterial consortia. Using the same format assigned to FIG. 3, the biocolloid 21 is positioned within the flow passageway and positioned through the naturally impressed anodic charges into the material of the wall 3. This biocolloidal growth is held in position by the relatively slow movement of the culturing fluid through the growing chamber and would show zones 22 with a greater density where the integrated bacterial consortial activity becomes observably denser.

The generation of biocolloidal structures involves the inoculation of the apparatus with the defined bacterial consortia in a process that allows effective interactions to occur under suitable conditions including the application of chemical feeds to assure the formation of the biocolloid with stimulation by such chemicals as phosphorus, nitrogen, polysaccharides, calcium and carbon dioxide to allow effective synthesis of the desired growth at sites created by the natural impression of anodic charges into the walls 2, 3 of the flow passageways P.

### 2. METHODOLOGY

Methods associated with the apparatus described in FIGS. 1 and 2 involve different methods being applied to the integrated bacterial consortially cultured structures described in FIG. 3 for the formation of bioconcretions and in FIG. 4 for the formation of biocolloidal structures to maximize the production of the desired product.

The apparatus described in FIGS. 1-4 relies upon the naturally induced electrical potentials for the impression of some of the electrically conductive surfaces within the apparatus with anodic charges directly as a result of the application of the selected bacterial consortia to the apparatus once saturated with the commonly water-based fluids that serve as the support medium for the interactive growth and activities of these consortia. The admission of the consortia to the apparatus leads to an electromagnetically naturally induced charge gradient across the culturing fluid between the deployed surfaces that also forms a focal point for naturally induced electrolytic functions to occur with significant impacts on the oxidation-reduction gradients as both oxygen and hydrogen are generated as products from this electrolytic function.

Methodologies that are unique to this disclosure relate to the practice of employing a multiplicity of defined bacterial consortia that are inoculated in a sequence that maximizes the potential for the formation of the bioconcretious or biocolloidal structure in a manner that ensures production of the desired end product. Industrial microbiological processes and events are commonly driven by either inoculations of a single desired species, or a single application per batch of some naturally derived inoculum rich in the microbial activities of choice. The methodology of this disclosure utilizes the described apparatus followed by the addition of suitable nurturing fluids to cultivate the activities, that is then followed by the sequential addition of the bacterial consortia of choice. As the innocula are added, the fluid flow is initiated within the apparatus at the defined operating conditions required for the form of growth and the nature of the multiplexed consortial activity to be undertaken. Table One indicates a sequence for the inoculation of the apparatus for the generation of bioconcretious (middle column) and biocolloidal (right-hand column) growths.

TABLE ONE
Methodology sequence for operation of the apparatus

| | Bioconcretious | Biocolloidal |
|---|---|---|
| Factor added | growth (BCN) | growth (BCL) |
| Sterile distilled water | Saturation of apparatus | Saturation of apparatus |
| (A) | and all lines | and all lines |
| Flow (B) | 2 to 5 volumes/hr | 0.1 to 1 volume/hr |
| Temperature (C) | 28 ± 2° C. | 36 ± 1° C. |
| pH amended (D) | 7.2 to 8.4 range | 6.8 to 7.6 range |
| Medium type (D) | BCN 10% by volume | BCL 5% by volume |
| IRB-(CL, BC, BR)* | 4% volume added (1) | 2% volume added (5) |
| SRB-(BT-BB)* | 2% volume added (2) | 1% volume added (4) |
| HAB-(UP)* | 1% volume added (4) | 5% volume added (1) |
| SLYM-(CL-SR)* | 3% volume added (3) | 4% volume added (2) |
| DN-(FO)* | 1% volume added (5) | 1% volume added (3) |

Note:
Bracketed letters in the left hand column indicate sequence of application and the asterisk (*) indicates the order these ingredients are added given numerically in the middle and right hand columns;
volume is defined as the total saturation volume for the apparatus that can be filled with culturing fluids.

Two cultural media are employed. BCN medium is designed for the generation of bioconcretions within the apparatus while BCL is designated for the stimulation of biocolloids. The precise formulations for these media would be affected by the precise nature of the bacterial consortia being inoculated into the apparatus. BCN media would carry high concentrations of iron commonly in the ferrous form, citrate, sulfate, mono- and dibasic phosphate, ammonium and nitrate nitrogen, potassium sulfate and the normal range of micro-nutrients for bacterial activity. BCL media is primarily going to create stable biocolloidal masses within the fluid and so has a different formulation that would be dominated by carbohydrates including degradable polymeric forms as well as glucose and short chain fatty acids, dibasic phosphate, calcium carbonate, potassium chloride and sulfate, ammonium nitrate and the normal range of micronutrients for bacterial activity.

Process optimization involves two mechanisms as the primary driving forces in the suppression of pathogenic microbes by the consortial growths and/or that product. These mechanisms: (1) are members of, or the bacterial consortium as a whole, being able to out-compete any alien infesting microbial species that could invade and infest the functioning environment within the apparatus thus maintaining production efficiency with a minimum of lost production capacity due to these potential infestation effects; and (2) utilize the ability of the selected bacterial consortia to provide such an extreme, or attractive sorptive sites for the infesting alien microbes that these organisms are sorbed into the complex consortial matrix and neutralized. Such monitoring of the process can be achieved by well-known methods for microbial activity detection.

### 3. EXAMPLES

The following examples are based on various forms of consortial activity that can be monitored within the process that is the subject of this disclosure. The first example uses isolated bacterial consortia from iron-rich bioconcretious growths and were cultured using the BART testers. These consortia included five BART tester types and each generated a significant consistent reaction pattern signature (RPS). Descriptions of the RPS can be found in Cullimore, 1999. Table Three includes the standard RPS for each of the bacterial consortia that are cultured and the time length required for incubation at room temperature (21±1° C.) and the length of time the culture would remain active before senescence.

TABLE THREE
Descriptions of the five consortia by BART reaction

| Designation | BART type | RPS | Active time period |
|---|---|---|---|
| Iron related | IRB-BART | CL-BC-BR | 2 to 8 wks |
| Sulfate reducing | SRB-BART | BT-BB | 3 to 12 wks |
| Heterotrophic | HAB-BART | UP | 1 to 4 wks |
| Slime forming | SLYM-BART | CL-SR | 1 to 4 wks |
| Denitrifying | DN-BART | FO | 1 to 3 wks |

Note:
Designation refers to the common name used for the specific group of bacteria;
BART type is the standard nomenclature used to define the tester;
RPS is the distinctive reaction pattern signature that is generated by the consortia being cultured and it should be noted that different consortia can generate different RPS and would be considered atypical for this example; and
Active time period refers to the time period when the consortia would be considered the most active after the time of inoculation (at 2% volume/volume) and during incubation.

To initiate the use of the apparatus for the culture of the consortia, the following protocol can be effectively used to ensure that there is a formation of bioconcretious growths within the confines of the apparatus. Sequences in the protocol to assure growth includes the following steps: (1) saturate the apparatus with 4% seawater salt along with 0.4% nitrogen as sodium nitrate, and 0.2% phosphorus as potassium dihydrogen phosphate with the pH adjusted to 7.8; (2) allow the apparatus 48 hours for the generation of electrical charges between the applied metals and surfaces constructed within the apparatus; and (3) inoculate the suspended BART consortia to the total volume of recycling liquids in the apparatus in the following sequence and % volume/volume ratios: IRB, 5% followed by one hour wait; SRB, 2%; DN, 1%, SLYM, 2% followed by one hour wait; and finally the HAB, 5%. At this stage the liquids in the apparatus are recycled at a rate of three recycles per day. Interactions between the consortia with the nutrients, salts and impressed electrical charges within the apparatus causes the bioconcretions to form at focal sites within the apparatus in a manner that could be observed either directly, or through hydraulic effects causing the flow through of the recycling fluids to slow down because of biological occlusions forming within the recycling passageways.

In this example, these bioconcretions involving all of inoculated consortia should be formed at different locations after four weeks of incubation. Six weeks into incubation then there should now be production of a desirable chemical product. In the case of this example, the product would be a definable broad spectrum antibiotic (BSA) that would continue to be produced by the cooperative activities of the consortia for a period of eight weeks before the levels of production begin to decline. This example utilizes five different consortia of bacterial origin that have the joint ability to generate the BSA only when all five consortia are present and active within the apparatus.

Determination of the inhibitory nature of the BSA was determined specifically to a range of five American Type Culture Collection (ATCC) strains of bacteria (Pseudomonas aeruginosa, ATCC #27853; Staphylococcus epidermis, ATCC #12228; Escherichia coli, ATCC #25922; Serratia marcescens, ATCC #8100) and Proteus vulgaris, ATCC #13315). This example of the evaluation of the inhibitory effects of the consorm-generated product (designated BSA) used time lapses generated in seconds to the onset of detectable reductive conditions in the heterotrophic aerobic bacteria HAB-BART system. The time lag to a reductive state illustrated the level of microbial activity with shorter time lags meaning greater microbial activity and a lesser impact of the BSA on the bacterial activity. The results of these tests are summarized in Table Two.

TABLE TWO
BSA impacts on selected ATCC genera of bacteria

| ATCC # | Genus | Population | Average % change |
|---|---|---|---|
| 25923 | Staphylococcus | 78 × 10⁶ | −92% |
| 14028 | Salmonella | 4.7 × 10⁹ | −55% |
| 13315 | Proteus | 6.1 × 10⁷ | −100% |
| 8106 | Serratia | 1.4 × 10⁹ | −87% |
| 27853 | Pseudomonas | 15 × 10⁶ | +900% |

Note:
populations are in predicted active cells/ml using the BART-READ system with five replicates;
average percentage change is based upon the five replicate analyses where a negative indicates the percentage reduction in population due to the BSA as an inhibitory effect and positive indicates a stimulatory effect.

In these trials the BSA was found to have a total inhibition of Proteus; a significant inhibitory effect of at least one order of magnitude on Staphylococcus and a stimulatory effect on Pseudomonas. This trial set confirms that a combination of five consortia within the apparatus and using the method of this disclosure are jointly capable of generating an antibiotic with determinable inhibitory characteristics.

As a second example, the effectiveness of the process could be determined by the generation of a BSABC from a biocolloidal complex created within the apparatus as described above but where the metal alloys were replaced with carbon-based materials. These caused biocolloidal flocs to form in the fluid passageways rather than bioconcretious growth structures. BSABC when extracted as a dissolved extract using 0.22 micron filtration was found to have a completely inhibitory effect on Staphylococcus epidermis (ATCC strain 12228), suppressing cell populations by greater than five orders of magnitude.

Accordingly, the present invention is therefore based on the demonstrated fact that microbial activities can take place by synergistic interactions between various bacterial consortia that each involves several species that are co-dependent. These consortia therefore include mutually interdependent mechanisms that serve to protect the collective consortia from infestation by alien species that are potentially hostile to the functioning of the integrated bioconcretious or biocolloidal environments dominated by the consortial activities.

A prime focus of the present invention is therefore that these consortially driven defense mechanisms can be utilized to exploit a valuable array of manageable chemicals and processes that could then be harnessed to reduce and eliminate infestations, infection, bacterial and cancerous growths in humans.

While the present invention has been described with reference to presently preferred embodiments thereof, other embodiments as well as obvious variations and modifications to all the embodiments will be readily apparent to those of ordinary skill in the art. The present invention is intended to cover all such embodiments, variations and modifications that fall within the spirit and scope of the appended claims.

What is claimed is:

1. An apparatus for growing bioconcretious or biocolloidal structures from a culturing fluid of bacterial consortia, the apparatus comprising: a growing chamber having an inlet side that admits a culturing fluid of bacterial consortia into the growing chamber, an outlet side that discharges the culturing fluid from the growing chamber, and a plurality of flow passageways that connect the inlet side to the outlet side, the flow passageways having along their lengths alternate constricted and expanded flow regions that alternately constrict and expand the flow of culturing fluid therealong to promote growth of bioconcretious or biocolloidal structures within the culturing fluid or on surface portions of the flow passageways.

2. An apparatus according to claim 1; wherein the flow passageways each have opposed spaced-apart electrically conductive surfaces that interact with the bacterial consortia in the culturing fluid to generate an electric field to promote the growth of bioconcretious or biocolloidal structures.

3. An apparatus according to claim 2; wherein the opposed electrically conductive surfaces are comprised of different metals.

4. An apparatus according to claim 2; wherein the opposed electrically conductive surfaces are comprised of carbon-based materials.

5. An apparatus according to claim 1; wherein the flow passageways have spaced-apart opposed walls that extend in a flow direction from the inlet side to the outlet side and that are alternately spaced closer to and farther from one another to define the alternate constricted and expanded flow regions.

6. An apparatus according to claim 5; wherein the opposed walls of the flow passageways are comprised of different metals.

7. An apparatus according to claim 5; wherein the opposed walls of the flow passageways are comprised of carbon-based materials.

8. An apparatus according to claim 5; wherein each two adjoining flow passageways share a common wall.

9. An apparatus according to claim 1; further including a control vessel having an inlet connected to the outlet side of the growing chamber and an outlet connected to the inlet side of the growing chamber, an outlet port through which culturing fluid can be extracted from the control vessel, and an inlet port through which additives and supplemental culturing fluid can be introduced into the control vessel, whereby culturing fluid can be continuously recycled through the apparatus while desired quantities of culturing fluid are extracted and supplemental culturing fluid and additives are introduced.

10. An apparatus according to claim 9; further including a collection conduit that is connected to collect the culturing fluid from the outlet side of the growing chamber and deliver it to the inlet of the control vessel, and a return conduit that is connected to return culturing fluid and additives from the outlet of the control vessel to the inlet side of the growing chamber.

11. A method of growing bioconcretious or biocolloidal structures from a culturing fluid of bacterial consortia comprising the steps:
providing a culturing fluid of preselected bacterial consortia; and
flowing the culturing fluid between two spaced surfaces constructed of different metals or carbon-based materials so that the bacterial consortia interact with the surfaces to generate an electrically charged field effective to create growths of bioconcretious or biocolloidal structures within the culturing fluid and on one of the surfaces.

12. A method according to claim 11; wherein the two spaced surfaces are alternately spaced closer together and farther apart along the flow direction of the culturing fluid.

13. A method according to claim 11; wherein the flowing step comprises flowing the culturing fluid between a plurality of pairs of spaced surfaces arranged in parallel flow relationship, each pair of spaced surfaces being constructed of different metals or carbon-based materials so that the bacterial consortia interact with the surfaces to generate an electrically charged field effective to create growths of bioconcretious or biocolloidal structures within the culturing fluid and on one of the surfaces.

14. A method according to claim 13; further including the step of adding one or more additives to the culturing fluid to assure creation of growths of biocolloidal structures.

15. A method according to claim 14; wherein the one or more additives comprise one or more of phosphorous, nitrogen, polysaccharides, calcium and carbon dioxide.

16. A method according to claim 13; further including the step of adding one or more additives to the culturing fluid to assure creation of growths of bioconcretious structures.

17. A method according to claim 16; wherein the one or more additives comprise one or more of ferrous iron, calcium and carbon dioxide.

18. A method according to claim 13; wherein the surfaces of each pair of spaced surfaces are alternately spaced closer together and farther apart along the flow direction of the culturing fluid so that growths of bioconcretious or biocollodial structures occur on one of the surfaces at sites where the spaced surfaces are closer together.

19. A method according to claim 11; further including the step of adding one or more additives to the culturing fluid to assure creation of growths of biocolloidal structures.

20. A method according to claim 11; further including the step of adding one or more additives to the culturing fluid to assure creation of growths of bioconcretious structures.
